(19) United States
(12) Patent Application Publication
(10) Pub. No.: US 2026/0196340 A1
(43) Pub. Date: Jul. 9, 2026

(54) SURGICAL INVENTORY CONTROL SYSTEM

(71) Applicant: UNIVERSITY OF SHARJAH, SHARJAH (AE)

(72) Inventors: Ihab FOUAD, Sharjah (AE); Safaa ALMELHEM, Sharjah (AE); Dani AL ASHKAR, Sharjah (AE)

(21) Appl. No.: 19/008,867

(22) Filed: Jan. 3, 2025

(51) Int. Cl. G16H 40/20

(52) U.S. Cl. CPC G16H 40/20

(57) ABSTRACT

A surgical inventory control system comprising a surgical board. The surgical board comprises a plurality of receptacles and a plurality of surgical instruments configured to be received within the plurality of receptacles. The surgical board detects whether each surgical instrument is received within a respective receptacle of the plurality of receptacles via a tracking device. The system also comprises a surgical bed comprising a plurality of sensors arranged to detect the respective tracking device of each surgical instrument when in use on the surgical bed. A user can be notified if a surgical instrument is not returned to the surgical board, and of the position of the surgical instrument on the surgical bed.

### FIELD

The present disclosure concerns surgical inventory control systems. More particularly but not exclusively, the present disclosure concerns a surgical inventory control system comprising a surgical board that detects whether each surgical instrument is received within a respective receptacle of the surgical board.

### BACKGROUND

Background description includes information that will be useful in understanding the present invention. It is not an admission that any of the information provided herein is prior art or relevant to the presently claimed invention, or that any publication specifically or implicitly referenced is prior art.

Typically, a supporting medical practitioner such as a nurse places the surgical items such as scalpels, scissors, and gauze on a sterile table for the surgeon to use.

Nurses in the operating theatre typically bring gauze to a table and count them manually, then placing them on a surgical board. Due to stress, fatigue, and increased demands in the operating theatre; nurses tend to forget and make mistakes while counting. This may result in misplacement of surgical items or contamination, or even more seriously, may result in surgical items being left in the patient once the operation is complete.

The cases of retained surgical instruments has been increasing over the years. Forgetting an instrument in a patient can cause a prompt immune reaction and cause bacterial dissemination. There are instances where patients who have been treated by relatively simple surgeries have died from forgotten sponges and not from the surgery or the disease itself.

Health care workers have attempted to solve this issue by manually counting the sponges and other instruments; however, this has not mitigated the frequency of retained surgical instruments with the number of cases still rising. Human error, stress, fatigue, multitasking are human factors that influence medical practitioners'ability to adhere fully to counting requirements in the operating room (OR).

The present disclosure seeks to mitigate one or more of the abovementioned problems. More specifically, but not exclusively, the present disclosure seeks to provide an improved surgical inventory control system.

### SUMMARY

According to a first aspect of the present disclosure there is provided a surgical inventory control system. The surgical inventory control system comprises a surgical board. The surgical board comprises a plurality of receptacles and a plurality of surgical instruments configured to be received within the plurality of receptacles. Each surgical instrument of the plurality of surgical instruments comprises a tracking device such that the surgical board detects whether each surgical instrument is received within a respective receptacle of the plurality of receptacles. The surgical inventory control system comprises a surgical bed. The surgical bed comprises a plurality of sensors arranged to detect the respective tracking device of each surgical instrument when in use on the surgical bed. A notification is issued to a user of the surgical board if a surgical instrument of the plurality of surgical instruments is not received within its respective receptacle. The user can be notified of a position of the surgical instrument on the surgical bed.

Advantageously, the surgical board is able to inform the user of the board, who may be a surgeon operating within an operating theatre, which instruments are not present within their respective receptacles of the surgical board. Additionally, for the instruments that are not received within their respective receptacles, the surgical inventory control system is able to determine whether those instruments are located in a position above the surgical bed. By using an inventory control system according to the present disclosure, the user forgoes the need for manual counting of instruments and eliminates the risk of leaving instruments inside the patient.

The tracking of the instruments on the surgical bed speeds up the process of locating potentially small instruments inside the patient in the event that an instrument has been mistakenly forgotten by the surgeon.

The surgical board may comprise light indicators to indicate whether the respective instrument is received within its respective receptacle. The light may indicate a green color to indicate presence of the surgical instrument within its respective receptacle. The light may indicate a red color to indicate that the surgical instrument is not present within its respective receptacle.

Each receptacle may comprise a sensor. The sensor may be a pressure sensor. The pressure sensor may detect the presence of a surgical instrument within the receptacle. The sensor may be a radio frequency identification (RFID) sensor.

The tracking device may comprise a radio frequency identification (RFID) tag.

RFID tags are harmless to patients as they are non-toxic and do not emit harmful radiation that may have an adverse effect on the patient. RFID tags are also capable of being tracked precisely in three-dimensions by a plurality of sensors, ensuring that the tagged surgical instruments can be located quickly by the surgical inventory control system. RFID tags are advantageously also passive, and do not require any charging or on-board power.

The surgical bed may comprise three or more sensors.

Having at least three sensors enables the system to accurately determine the location of an RFID tag in 3D space.

The surgical bed may comprise four or more sensors.

Having at least four sensors increases the precision with which the system can determine the special location of the respective RFID tag of each instrument.

During a surgical operation, the surgical board may create timestamps associated with removal and replacement of each surgical instrument from its respective receptacle, thereby creating a time-based record of when the surgical instruments are removed from and replaced in their respective receptacles.

By creating a report of usage of the surgical instruments, the surgical inventory control system ensures integrity of the surgical instruments, and helps users, or surgeons, to improve their or their colleagues'adherence to surgical operating procedures.

The surgical inventory control system may comprise a gauze dispenser.

The gauze dispenser may count the number of gauzes dispensed during a surgical operation.

The gauze dispenser may display the number of gauzes dispensed during a surgical operation.

The surgical inventory control system may comprise a disposal container. The disposal container may count the number of gauzes disposed therein. The disposal container may count the number of gauzes by use of a sensor.

The gauzes may comprise a tracking device.

By counting the number of gauzes dispensed, and the number disposed, the system is able to determine whether any gauzes have been left within the operating theatre or potentially within the patient. This would advantageously inform the user that they need to account for one or more missing gauzes in the operating theatre or within the patient.

The gauze dispenser may be voice activated.

The surgical inventory control system may comprise a sponge dispenser.

The sponge dispenser may count the number of sponges dispensed. The sponge dispenser may count the number of sponges dispensed during a surgical operation.

The sponge dispenser may display the number of sponges dispensed during a surgical operation.

The surgical inventory control system may comprise a disposal container. The disposal container may count the number of sponges disposed therein. The disposal container may count the number of sponges by use of a sensor.

Each sponge may comprise an RFID tag.

The sponge dispenser may comprise an RFID reader. The RFID reader may count the number of sponges dispensed by the sponge dispenser.

The disposal container may count the sponges by detecting the sponge's RFID tag via a sensor as it enters the disposal container.

The disposal container may be the same disposal container as the gauze disposal container.

The disposal container may comprise a proximity sensor and an RFID sensor. The RFID sensor may be an RFID reader.

By having both a proximity sensor and an RFID reader, the disposal container is able to count and differentiate between both gauzes and sponges that are disposed therein. Therefore, only a single disposal container would be needed while maintaining integrity of the inventory control system.

The disposal container may be comprised in the surgical board. The disposal container may be separate from the surgical board. The disposal container may be separate from the surgical board, and in communication with the surgical board.

The surgical inventory control system may comprise a waste container that records the number of sponges disposed therein, by detecting the respective RFID tags of each sponge disposed therein.

The surgical inventory control system may comprise a sensor configured to detect whether a person is within a predefined distance to the surgical board.

The sensor may comprise a light detection and ranging (Lidar) sensor.

The sensor may comprise a camera.

The lidar sensor is advantageously able to determine position and range of multiple objects (including people) in a spatial field.

The sensor may differentiate between authorized users of the surgical board, and unauthorized users of the surgical board. The surgical inventory control system may issue a warning when an unauthorized user of the surgical board is less than the predefined distance from the surgical board.

By identifying authorized users and issuing a warning when unauthorized users are too close to the surgical board, the inventory control system maintains integrity of the surgical inventory by ensuring that only authorized people are allowed to be within a predetermined distance to the surgical board. By identifying authorized users and issuing a warning when unauthorized users are too close to the surgical board, the inventory control system also minimizes the risk of contamination of the surgical instruments by unauthorized persons.

According to a second aspect of the present disclosure, there is provided a method of controlling an inventory of surgical instruments. The method comprises detecting, via a tracking device comprised in a surgical instrument, when the surgical instrument has been removed from a receptacle of a surgical board. The method comprises tracking the location of the surgical instrument on a surgical bed, the surgical bed comprising a plurality of sensors arranged to detect the position of the tracking device. The method comprises detecting, via the tracking device, when the surgical instrument has been returned to the receptacle. The method comprises notifying a user of the surgical board if the surgical instrument has not been returned to the receptacle.

The method may comprise identifying authorized users of the surgical board; and issuing a warning when an unauthorized user of the surgical board is within a predetermined distance from the surgical board.

A distance of the unauthorized user may be determined using a camera.

A distance of the unauthorized user may be determined using lidar.

The method may comprise dispensing gauze from the surgical board; and counting a number of gauze dispensed from the surgical board.

The method may comprise dispensing a sponge from the surgical board, the sponge comprising a tracking device.

The method may comprise counting a number of sponges disposed within a waste container via the tracking device.

The tracking device may comprise a radio frequency identification (RFID) tag.

The method may comprise associating a removal timestamp with when the surgical instrument has been removed from its receptacle; associating a returning timestamp with when the surgical instrument has been returned to its receptacle; and creating a time-based record of when the surgical instrument has been removed from and replaced in its receptacle.

It will be understood that features and advantages disclosed in relation to one aspect of the present disclosure may be applicable to another aspect of the present disclosure, and vice versa.

### BRIEF DESCRIPTION OF THE DRAWINGS

The manner in which the above-recited features of the present invention is understood in detail, a more particular description of the invention, briefly summarized above, may be had by reference to embodiments, some of which are illustrated in the appended drawings. It is to be noted, however, that the appended drawings illustrate only typical embodiments of the present disclosure and are therefore not to be considered limiting of its scope, for the present disclosure may admit to other equally effective embodiments.

FIG. 1A shows a surgical inventory control system according to an embodiment of the present disclosure.

FIG. 1B shows a display of a surgical board according to an embodiment of the present disclosure.

FIG. 1C shows a sponge according to an embodiment of the present disclosure.

FIG. 2 shows a surgical board according to an embodiment of the present disclosure.

FIG. 3 shows a surgical inventory control system according to an embodiment of the present disclosure.

FIG. 4 shows a method of controlling an inventory of surgical instruments according to an embodiment of the present disclosure.

The foregoing and other objects, features and advantages of the present invention, as well as the invention itself, will be more fully understood from the following description of preferred embodiments, when read together with the accompanying drawings.

### DETAILED DESCRIPTION

The present disclosure relates to the field of surgical inventory control systems, and more particularly to a surgical inventory control system comprising a surgical board that detects whether each surgical instrument is received within a respective receptacle of the surgical board.

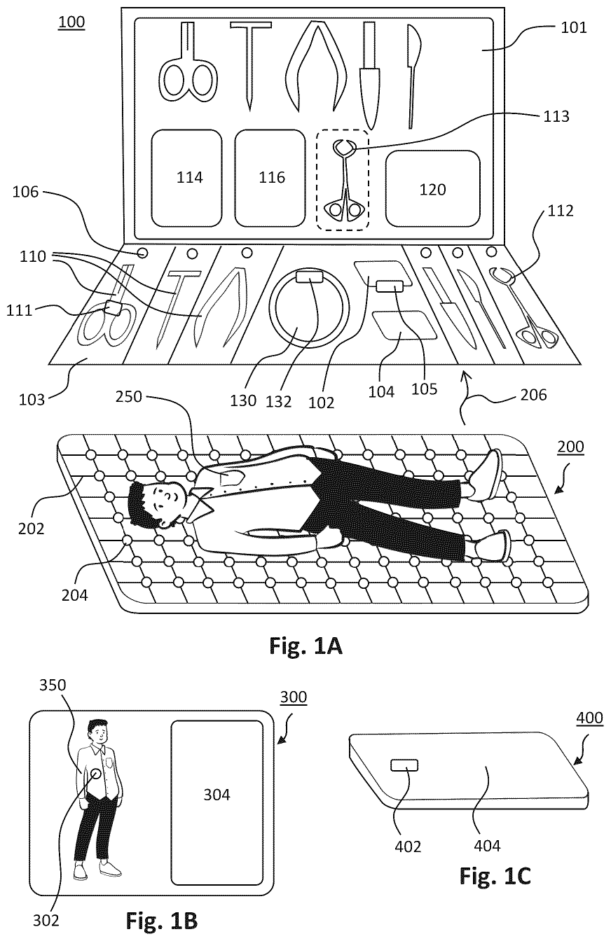

The principles of the present invention and their advantages are best understood by referring to FIG. 1 to FIG. 4. In the following detailed description of illustrative or exemplary embodiments of the disclosure, specific embodiments in which the disclosure may be practiced are described in sufficient detail to enable those skilled in the art to practice the disclosed embodiments. The following detailed description is, therefore, not to be taken in a limiting sense, and the scope of the present disclosure is defined by the appended claims and equivalents thereof. References within the specification to “one embodiment,” “an embodiment,” “embodiments,” or “one or more embodiments” are intended to indicate that a particular feature, structure, or characteristic described in connection with the embodiment is included in at least one embodiment of the present disclosure.

FIG. 1A shows a surgical inventory control system according to an embodiment of the present disclosure.

The surgical inventory control system comprises a surgical board 100 and a surgical bed 200.

The surgical board 100 has a number of receptacles 103 that house various surgical instruments 110 such as scissors, pliers, knives, scalpels, tweezers, and surgical cutters. When each of the surgical instruments 110 are received within their respective receptacle 103 of the surgical board 100, the light indicator 106 for the respective receptacle is illuminated in green.

When a surgical instrument 103 is not received in its respective receptacle 103, the light indicator 106 is illuminated red. This gives a visual indication to the surgeon and any nurses in the operating theatre that a surgical instrument 103 has been removed from the surgical board 100, and to ensure once the operation is complete that all surgical instruments 103 have been accounted for.

Each surgical instrument 103 has a radio frequency identification (RFID) tag 111 (only shown on one instrument in FIG. 1 for clarity purposes).

In embodiments, the surgical board detects the presence of the surgical instrument within its respective receptacle by detection of the RFID tag via one or more RFID readers located on or within the surgical board.

In embodiments, the surgical board detects the type of surgical instrument received within its respective receptacle by detection of the RFID tag via one or more RFID readers located on or within the surgical board.

In this way, the surgical board may also detect whether the wrong surgical instrument has been placed into a receptacle intended for a different surgical instrument. The light indicator may be illuminated in a different color to indicate to the surgeon or nurses that the wrong surgical instrument has been placed within the receptacle. The light indicator may be illuminated in orange, for example, to indicate to the surgeon or nurses that the surgical instrument has been placed in a receptacle intended for a different surgical instrument.

The surgical board 100 also comprises a display 101. The display 101 provides a visual indication of the surgical inventory.

If an instrument, such as a surgical cutter 112 is removed from the surgical board, the display 101 provides a digital indication 113 on the screen that the surgical cutter has been removed. The digital indication in embodiments is in the form of a border around an image of the instrument that is removed. In this example, a dashed border around the removed surgical cutter indicates that the surgical cutter is missing from the surgical board.

In embodiments, removed or missing items or instruments are marked with a red border on the screen 101.

The screen 101 also displays other information. A number of dispensed sponges 114 is indicated on the screen 101. A number of dispensed gauzes 116 is indicated on the screen. An image 120 of where an instrument is located on the surgical bed 200 is also indicated on the screen 101.

The surgical board 100 also comprises a sponge dispenser 102. The sponge dispenser 102 is voice activated. In embodiments, the sponge dispenser is only activated by the voice of authorized users. The sponge dispenser 102 counts the number of sponges dispensed during an operation.

The sponge dispenser comprises an RFID reader 105. The sponges dispensed comprise an RFID tag (see FIG. 1C). The RFID reader 105 counts the number of sponges dispensed by

In embodiments, the sponge dispenser 102 is a bucket containing a plurality of sponges, each sponge comprising an RFID tag. The surgeon may reach into the bucket and manually retrieve as many sponges as necessary, with the RFID reader capable of detecting the removal of each sponge, thereby providing an accurate count of the number of sponges removed.

Each time a sponge is removed from the sponge dispenser 102, the count 114 increases by one, indicating the total number of sponges currently in circulation.

The surgical board 100 also comprises a gauze dispenser 104. The gauze dispenser 104 is voice activated. In embodiments, the gauze dispenser is only activated by the voice of authorized users. The gauze dispenser 104 counts the number of gauzes dispensed during an operation.

In embodiments, the surgical board comprises a button that activates either the sponge dispenser or the gauze dispenser, causing them to dispense one or more sponges or gauzes.

The gauze dispenser may dispense one gauze at a time, so that counting the number of gauzes dispensed can be done accurately.

The inventory control system comprises a disposal container 130. In the embodiment of FIG. 1A, the disposal container is comprised in the surgical board 100.

In embodiments, the disposal container is separate from the surgical board. In embodiments, the disposal container is separate from the surgical board and in communication with the surgical board.

The disposal container 130 comprises an RFID reader 132. The RFID reader 132 detects when sponges are disposed therein. When the RFID reader 132 of the disposal container 130 detects that a sponge has been disposed therein, the count of sponges 114 is reduced by one, thereby indicating accurately the number of sponges currently in circulation.

The disposal container also comprises a proximity sensor. The proximity sensor is configured to detect the disposal of gauzes. When the proximity sensor detects disposal of an object, but there is no detection by the RFID reader, then that would indicate that gauze has been disposed. Each time gauze is dispensed by the gauze dispenser 104, the count 116 increases by one. Each time the disposal container detects that gauze has been disposed of the count 116 decreases by one, thereby indicating the number of gauzes currently in circulation.

The surgical inventory control system also comprises a surgical bed 200. For illustrative purposes, a figurative illustration of a patient 250 is shown lying on the surgical bed 200.

The surgical bed comprises a plurality of sensors 204 arranged in a grid pattern 202.

The sensors are RFID readers, configured to detect the RFID tags of the various surgical instruments and sponges.

By having at least three RFID readers, the surgical bed is able to communicate 206 with the surgical board 100 to indicate the spatial location of each surgical instrument or sponge on the surgical bed. This is displayed 120 on the screen 101 of the surgical board for ease of reference by the surgeon or other users in the operating theatre.

FIG. 1B shows a display of a surgical board according to an embodiment of the present disclosure.

The display 300 may be included on the screen 101 of the surgical board 100, for example in location 120. The display 300 includes a representation of the patient 350 and illustrates a location 302 of where a surgical instrument is currently located on the surgical bed. This enables the surgeon to locate the surgical instrument quickly and accurately on the surgical bed and inside the patient to minimize the time taken for inventory and minimize any potential risk of harm to the patient by extended exposure to the presence of surgical instruments inside their body.

The display 300 also includes text 304 comprising additional information for the surgeon. The text may include information comprising a list of instruments currently located on the surgical bed, and when they were removed from the surgical board, for example.

During the operation, the surgical board 100 associates a removal timestamp with when each surgical instrument has been removed from its receptacle. The surgical board also associates a returning timestamp with when each surgical instrument has been returned to its respective receptacle.

At the end of an operation, the surgical board creates a time-based record of when each surgical instrument has been removed from and replaced in its receptacle, enabling accurate logging of each surgical instrument for inventory and accurate due diligence.

FIG. 1C shows a sponge 400 according to an embodiment of the present disclosure.

The sponge 400 comprises a sponge body 404 comprised of a spongy absorbent material. The sponge also comprises an RFID tag 402.

The RFID tags are passive, requiring no on-board power. The RFID tags activate in response to incoming radio frequencies emitted by an RFID reader, causing them to reflect their own unique ID code, enabling accurate identification of each piece of surgical equipment.

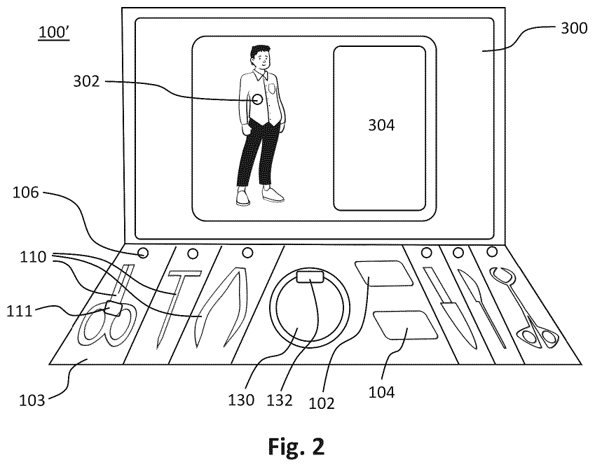

FIG. 2 shows a surgical board 100′ according to an embodiment of the present disclosure.

In the embodiment of FIG. 2, like reference numerals denote like features. Features that are substantially the same as those described in relation to FIG. 1A will not be repeated for clarity and conciseness.

The surgical board 100′ only displays the screen 300, which includes a graphic representation of the patient on the surgical bed, and a text-based output 304. Superimposed on the graphic of the patient is an indication 302 of where a surgical instrument is currently located on the patient.

The text-based output 304 includes information concerning surgical instruments that are currently removed from their respective receptacles, and surgical instruments that are currently located on the surgical bed.

The text-based output may also include logs of when surgical instruments have been removed and returned to the surgical board, enabling real-time auditing by the surgeon or other authorized users in the operating theatre of the surgical instruments.

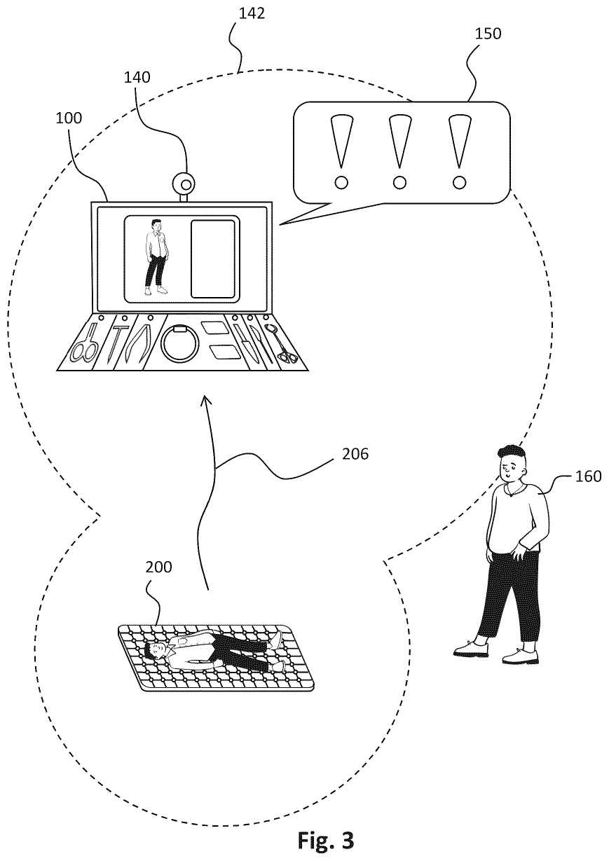

FIG. 3 shows a surgical inventory control system according to an embodiment of the present disclosure.

The surgical inventory control system comprises a surgical board 100, such as the surgical board of FIG. 1A or FIG. 2.

The surgical board also comprises a sensor 140. The sensor is an AI powered camera 140.

In embodiments, the sensor uses light detection and ranging (lidar). In embodiments, the sensor comprises a lidar sensor. In embodiments, the lidar sensor is AI powered.

The AI powered camera 140 is preconfigured to identify authorized users of the surgical board 100 and the surgical bed. The camera 140 is also preconfigured to detect whether a person 160 is within a predefined distance 142 to the surgical board.

The camera 140 differentiates between authorized users of the surgical board and unauthorized users of the surgical board, and the surgical inventory control system issues a warning 150 when an unauthorized user 160 of the surgical board is less than the predefined distance 142 from the surgical board.

The warning is an audio warning 150 projected by speakers within the surgical board. In embodiments, the warning is a visual warning on the screen of the surgical board. The warning will alert individuals to keep a safe distance from the apparatus. The presence of an alert system will also encourage users to be more careful and more vigilant in their movements around the operating theatre, ensuring safety and sterility protocols are followed.

The predefined distance may be preconfigured by the surgeon. The predefined distance may be preconfigured by an administrator.

The predefined distance is considered a sterile field, whereby only authorized users are permitted within the sterile field to ensure sterile integrity of the surgical board 100, the surgical instruments, and the surgical bed 200.

As part of the report generated by the surgical board, in addition to the log of instrument use, the report may also comprise when the sterile field was breached. The report may also comprise who breached the sterile field. In the case where the operating theatre has medical students present, the report may assist the medical practitioners in teaching and training the students in following medical protocol.

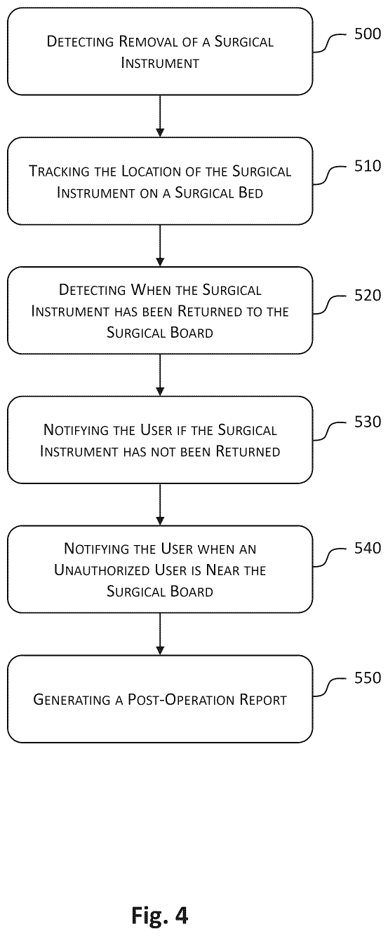

FIG. 4 shows a method of controlling an inventory of surgical instruments according to an embodiment of the present disclosure.

In a first step 500, the surgical board detects that a surgical instrument has been removed from its respective receptacle. This is done by detection of the RFID tag associated with each surgical instrument. An RFID reader located on/in the surgical board reads the RFID tags of the respective surgical instruments to determine whether they are received within their respective receptacles or not.

When the surgical instrument has been removed from the receptacle, a timestamp associated with the removed surgical instrument is created and logged.

The location of the surgical instrument is tracked 510 on a surgical bed. The surgical bed comprises a plurality of sensors arranged to detect the position of the RFID tag of each respective surgical instrument.

The system detects when the surgical instrument has been returned to the receptacle 520. Again, this is through the use of RFID readers located on/in the surgical board, reading the RFID tags associated with each surgical instrument. In embodiments, the surgical board may comprise additional sensors to detect the presence of an instrument in a receptacle, such as pressure sensors, weight sensors, or proximity sensors, for example.

When the surgical instrument is returned to the receptacle, a timestamp associated with the returning of the surgical instrument is created and logged.

If the surgical instrument has not been returned to the receptacle, then the user is notified 530. The user, in embodiments, is the operating surgeon. The notification takes the form of a light indicator on the surgical board indicating that an instrument has not been returned to that particular receptacle. The notification may also take the form of a graphical notification on a screen. The screen may be connected to the surgical board. The graphical notification may show the name or an image of the surgical instrument that has not been returned. The graphical notification may also show the time that the surgical instrument was removed and, if present on the surgical bed, the location of the instrument on the surgical bed.

The system also notifies the user when an unauthorized user is near the surgical board 540. An AI powered camera distinguishes between authorized users and unauthorized users and determines whether an unauthorized user has positioned themselves at a distance to the surgical board that is less than a predetermined distance.

The AI powered camera may also determine whether an unauthorized user has positioned themselves at a distance to the surgical bed that is less than a predetermined distance.

The predetermined distance around the surgical board and the surgical bed defines a sterile field, within which unauthorized users are not permitted to enter. If an unauthorized user enters the sterile field, the system emits an auditory notification alerting all people in the operating theatre that the sterile field has been breached.

In embodiments, only the surgical board has a sterile field.

Finally, the system generates a post-operation report. The post-operation report comprises logs of when the surgical instruments were removed and returned. The post-operation report also comprises information concerning when the surgical field was breached, and by whom.

It will be apparent to those skilled in the art that various modifications and variations can be made in the present invention without departing from the spirit or scope of the inventions. Thus, it is intended that the present invention covers the modifications and variations of this invention provided they come within the scope of the appended claims and their equivalents. The disclosures and the description herein are intended to be illustrative and are not in any sense limiting the present disclosure, defined in scope by the following claims.

Many changes, modifications, variations and other uses and applications of the present disclosure will become apparent to those skilled in the art after considering this specification and the accompanying drawings, which disclose the preferred embodiments thereof. All such changes, modifications, variations and other uses and applications, which do not depart from the spirit and scope of the present disclosure, are deemed to be covered by the invention, which is to be limited only by the claims which follow.

1. A surgical apparatus comprising:
a surgical board comprising:
a plurality of receptacles; and
a plurality of surgical instruments configured to be received within the plurality of receptacles;
wherein each surgical instrument of the plurality of surgical instruments comprises a tracking device;
such that the surgical board detects whether each surgical instrument is received within a respective receptacle of the plurality of receptacles; and
a surgical bed comprising a plurality of sensors arranged in a grid pattern and configured to determine the spatial coordinates of the respective tracking device of each surgical instrument when in use on the surgical bed, wherein the plurality of sensors comprises at least three sensors, and wherein the surgical bed communicates with the surgical board to reconstruct a three-dimensional spatial location of each surgical instrument on the surgical bed based on data from the at least three sensors;
wherein a notification is issued to a user of the surgical board if a surgical instrument of the plurality of surgical instruments is not received within its respective receptacle; and
wherein the surgical board comprises a screen, wherein the screen comprises an illustration of where a surgical instrument of the plurality of surgical instruments is currently located on the surgical bed based on the reconstructed three-dimensional spatial location, such that the user can be notified of a position of the surgical instrument on the surgical bed.

2. The surgical apparatus according to claim 1, wherein the tracking device comprises a radio frequency identification (RFID) tag.

3. The surgical apparatus according to claim 1, wherein during a surgical operation, the surgical board creates timestamps associated with removal and replacement of each surgical instrument from its respective receptacle, thereby creating a time-based record of when the surgical instruments are removed from and replaced in their respective receptacles.

4. The surgical apparatus according to claim 1, wherein the surgical inventory control system comprises a gauze dispenser.

5. The surgical apparatus according to claim 4, wherein the gauze dispenser counts and displays the number of gauzes dispensed during a surgical operation.

6. The surgical apparatus according to claim 4, wherein the gauze dispenser is voice activated.

7. The surgical apparatus according to claim 1, wherein the surgical inventory control system comprises a sponge dispenser.

8. The surgical apparatus according to claim 7, wherein each sponge comprises an RFID tag.

9. The surgical apparatus according to claim 8, wherein the surgical inventory control system comprises a waste container that records the number of sponges disposed therein, by detecting the respective RFID tags of each sponge disposed therein.

10. The surgical apparatus according to claim 1, wherein the surgical inventory control system comprises a sensor configured to detect whether a person is within a predefined distance to the surgical board.

11. The surgical apparatus according to claim 10, wherein the sensor comprises a camera.

12. The surgical apparatus according to claim 10, wherein the sensor differentiates between authorized users of the surgical board, and unauthorized users of the surgical board;
wherein the surgical inventory control system issues a warning when an unauthorized user of the surgical board is less than the predefined distance from the surgical board.

13. A method of tracking an inventory of surgical instruments, the method comprising:
detecting, via a tracking device comprised in a surgical instrument, when the surgical instrument has been removed from a receptacle of a surgical board;
tracking the location of the surgical instrument on a surgical bed, the surgical bed comprising a plurality of sensors arranged in a grid pattern and configured to determine the spatial coordinates of the tracking device, wherein the plurality of sensors comprises at least three sensors, and wherein the surgical bed communicates with the surgical board to reconstruct a three-dimensional spatial location of the surgical instrument on the surgical bed based on data from the at least three sensors;
detecting, via the tracking device, when the surgical instrument has been returned to the receptacle;
notifying a user of the surgical board if the surgical instrument has not been returned to the receptacle; and
displaying, to the user, an illustration of where the surgical instrument is currently located on the surgical bed based on the reconstructed three-dimensional spatial location.

14. The method according to claim 13, wherein the method comprises:
identifying authorized users of the surgical board; and
issuing a warning when an unauthorized user of the surgical board is within a predetermined distance from the surgical board.

15. The method according to claim 14, wherein a distance of the unauthorized user is determined using a camera.

16. The method according to claim 13, wherein the method comprises:
dispensing gauze from the surgical board; and
counting a number of gauze dispensed from the surgical board.

17. The method according to claim 13, wherein the method comprises:
dispensing a sponge from the surgical board, the sponge comprising a tracking device.

18. The method according to claim 17, wherein the method comprises:
counting a number of sponges disposed within a waste container via the tracking device.

19. The method according to claim 13, wherein the tracking device comprises a radio frequency identification (RFID) tag.

20. The method according to claim 13, wherein the method comprises:
associating a removal timestamp with when the surgical instrument has been removed from its receptacle;
associating a returning timestamp with when the surgical instrument has been returned to its receptacle; and
creating a time-based record of when the surgical instrument has been removed from and replaced in its receptacle.
